(19) United States
(12) Patent Application Publication
(10) Pub. No.: US 2025/0195432 A1
(43) Pub. Date: Jun. 19, 2025

(54) METHODS FOR PREPARING AND CHARACTERIZING ZEIN-FUCOIDAN CONJUGATED NANO-LIPOSOME FOR PHYTOL ENCAPSULATION

(71) Applicant: Zhejiang Academy of Forestry, Xihu District (CN)

(72) Inventors: Yanbin WANG, Xihu District (CN); Liang HE, Xihu District (CN); Meirong GAO, Xihu District (CN); Yadan CHEN, Xihu District (CN); Yuchuan QIN, Xihu District (CN); Shaofei YUAN, Xihu District (CN); Jian ZHANG, Xihu District (CN); Qin LI, Xihu District (CN); Junwen CHENG, Xihu District (CN)

(21) Appl. No.: 18/886,182

(22) Filed: Sep. 16, 2024

(30) Foreign Application Priority Data
Dec. 15, 2023 (CN) 202311728682.3

(51) Int. Cl. A61K 9/127; A61K 31/045

(52) U.S. Cl. CPC A61K 9/1277; A61K 9/1271; A61K 31/045

(57) ABSTRACT

The present disclosure relates to a method for preparing and characterizing zein-fucoidan conjugated nanoliposomes for phytol encapsulation. The method of preparation includes a preparation of zein-fucoidan nanoparticles. This includes (i). dissolving zein in a 75% ethanol solution, and preparing a fucoidan solution by mixing 40 mL of ultrapure water with 200 mg of fucoidan, (ii). removing ethanol from a resulting mixture, and (iii) eliminating insoluble substances to collect the zein-fucoidan nanoparticles. The method further includes. preparation of nanoliposomes and preparation of biopolymer conjugated nanoliposomes. The zein-fucoidan nanoparticles and nanoliposomes are combined in a specific ratio to prepare the biopolymer conjugated nanoliposomes. In the present disclosure, Zein and fucoidan are utilized as a composite biopolymer to modify nanoliposomes loaded with phytol, thereby enhancing the performance of phytol nanoparticles.

### CROSS-REFERENCE TO RELATED APPLICATION

This patent application claims the benefit and priority of Chinese Patent Application No. 202311728682.3 filed with the China National Intellectual Property Administration on Dec. 15, 2023, the disclosure of which is incorporated by reference herein in its entirety as part of the present application.

### TECHNICAL FIELD

The present disclosure relates to a method for preparing and characterizing a zein-fucoidan conjugated nano-liposome for phytol encapsulation.

### BACKGROUND

Phytol (PYT) is a diterpene alcohol with the chemical name (2E,3,7R,11R,15)-tetramethyl-hexadecen-1-ol, widely found in many aromatic plants and in the unsaponifiable fraction of plant oils. Phytol is a C20 diterpenoid compound, structurally derived from geraniol through the reduction of three double bonds. Existing research has indicated that phytol has multiple potential health benefits for humans, such as its ability to inhibit the proliferation of cancer cells, being effective against human epidermoid carcinoma cells, leukemia cells, and human glioblastoma cells. Moreover, phytol possesses anti-inflammatory properties, alleviates joint swelling and pain, stimulates osteoblast differentiation, reduces obesity and related metabolic disorders, inhibits cellular senescence and melanin production, and provides neuroprotective effects. However, phytol has poor water solubility and stability, which affects its absorption rate in the intestinal wall and its entry into the systemic circulation, posing significant challenges to the development of phytol-related functional products.

Zein is a safe food ingredient extracted from corn endosperm, known for its good biocompatibility, biodegradability, and excellent self-assembly capability. It has been widely used to encapsulate hydrophobic compounds such as curcumin, quercetin, phytol, and resveratrol. However, the use of zein is hindered by its poor stability (sensitivity to pH, salt concentration, and temperature), low solubility, and weak mechanical properties. Researchers have been extensively exploring the complexation of zein with polysaccharides to effectively overcome these drawbacks by forming zein-polysaccharide particles. Fucoidan (Fu), extracted from brown algae, is a promising material for preparing zein-polysaccharide particles, due to its unique structure, in which the main chain is composed of the repeated sulfate L-fucose units with minor galactose, mannose and other monosaccharides on the branch.

Currently, the modification of liposomes using zein and polysaccharides to prepare composite nanoparticles is primarily aimed at enhancing the biocompatibility, biodegradability, and antioxidant activity of medium-chain terpenoid compounds, with limited research available. There have been no reported studies on the modification of nanoliposomes loaded with phytol using zein and fucoidan as composite biopolymers to improve the performance of phytol nanoliposomes.

### SUMMARY

An objective of the present disclosure is to overcome the aforementioned deficiencies in the prior art and to provide methods for preparing and characterizing a zein-fucoidan conjugated nanoliposome for phytol encapsulation.

The technical solution adopted to solve the above problems is as follows: a method for preparing a zein-fucoidan conjugated nanoliposome for phytol encapsulation, including steps of:

### (A) Preparation of Zein-Fucoidan Nanoparticles by:

i. dissolving zein in an ethanol solution with a 75% volume to volume ratio to prepare a zein stock solution with a 1% (w/v) weight to volume ratio; preparing a fucoidan solution by mixing 40 mL of deionized water with 200 mg of fucoidan, adjusting pH to 3.5; mixing 10 mL of the zein stock solution with the fucoidan solution and stirring at 1000 g for 60 minutes to obtain a mixture;
ii. removing ethanol from the mixture using a rotary evaporator, and diluting a resulting solution to 50 mL with water at pH 3.5; and
iii. centrifuging a diluted solution at 1500 g for 10 minutes to remove insoluble materials, and collecting a resulting colloidal particle dispersion, namely zein-fucoidan nanoparticles;

### (B) Preparation of a Nanoliposome by:

i. dissolving lecithin, cholesterol, and phytol in 10 mL of ethanol, stirring to ensure complete dissolution;
ii. removing the ethanol at 45° C. and 70 rpm with a rotary evaporator to form a thin film on a container surface;
iii. placing the thin film in an oven at 30° C. to dry overnight to completely remove the ethanol;
iv. hydrating the thin film with 20 mL of ultrapure water, stirring continuously at 700 rpm and 50° C. for 1 hour; after hydration, performing ultrasonication in an ultrasonic cell disruptor in an ice bath for 15 minutes to obtain nanoliposomes that are uniform; and
v. storing the nanoliposomes at 4° C. for later use; and

### (C) Preparation of Biopolymer-Conjugated Nanoliposomes by:

mixing the zein-fucoidan nanoparticles and liposomes in a molar ratio of zein to cholesterol of 1:20; using three methods for mixing, including magnetic stirring at 700 rpm for 3 hours, high-pressure homogenization at 80 bar for 3 cycles, and a combination thereof, and storing a resulting conjugated nanoliposomes at 4° C. for future use.

In some embodiments, the lecithin is sourced from egg yolk, with a molecular weight of 758.06 kDa and a purity greater than 80%, and the phytol has a purity of 95%.

In some embodiments, formulations obtained in this disclosure are designated as follows: P-ZF, P-NL, P-NL-ZF-S, P-NL-ZF-HPH, and P-NL-ZF-S-HPH, where P represents phytol, Z represents zein, F represents fucoidan, NL represents nanoliposomes, S represents magnetic stirring, and HPH represents high-pressure homogenization.

A method for characterizing the zein-fucoidan conjugated nanoliposome for phytol encapsulation includes the following assessments:

### (D) Particle Size and Zeta Potential Measurement by:

analyzing mean particle size, polydispersity index, and (potential of composite particles by dynamic light scattering and microelectrophoresis techniques using a zetasizer; and diluting a particle dispersion 10-fold with distilled water to mitigate a multiple scattering effect;

### (E) Encapsulation Efficiency (EE %) Analysis by:

centrifuging a suspension of nanoparticles at 24,200 g for 22 minutes at 4° C.; separating a supernatant from solid components, and washing solid components twice with a phosphate-buffered saline (PBS) at pH 7.4; extracting phytol from both the nanoparticles and the supernatant using n-hexane, then sonicating a mixture at 30° C. for 20 minutes, and conducting quantitative analysis using gas chromatography-mass spectrometer (GC-MS) to calculate the encapsulation efficiency;

### (F) GC-MS Assay by:

analyzing phytol content using a 7890A-5975C GC-MS, where an Agilent J&W DB-5 ms chromatographic column has a size of 30 m×0.25 mm×0.25 μm, high-purity helium gas is used as a carrier gas at a flow rate of 1.0 mL/min, an injector temperature is set to 250° C., and a sample volume is 4.0 μL, and a splitless injection mode is used; a temperature program is started at 100° C. for 2 minutes, then increased to 250° C. at a rate of 5° C./min, and held at 250° C. for 32 minutes; an ion source temperature of the mass spectrometer is maintained at 230° C. and a transfer line temperature is set to 280° C.; a mass range of 35-600 amu, an electron energy of 70 eV and a solvent delay time of 4 minutes are set; and an obtained chromatographic ion fragment spectrum with standard ion fragments is cross-referenced for qualitative analysis of a target compound;

A method for quantifying phytol includes weighing 0.0100 g of phytol, dissolving the phytol in n-hexane, and making up to volume in a 10 mL brown volumetric flask, diluting a solution with n-hexane to prepare standard solutions at concentrations of 1.00 mg/mL, 0.40 mg/mL, 0.20 mg/mL, 0.10 mg/mL, and 0.05 mg/mL, filtering the standard solutions through a 0.45 μm nylon membrane and analyzing by using GC-MS; calculating a linear regression equation for phytol standard samples: y=2E+09X+9E+06 (R²=0.9983) to determine an encapsulation efficiency of the phytol;

### (G) Transmission Electron Microscopy (TEM) Observation by:

observing a morphology of vesicles in optimized liposomes from various batches using a high-resolution transmission electron microscope, diluting each sample with distilled water after preparation and carefully placing a single droplet onto a carbon-coated copper grid before allowing the single droplet to dry; and inverting the copper grid onto a 2% phosphotungstic acid solution and negative staining in order to enhance visibility of lipid components; and

### (H) Fourier Transform Infrared Spectroscopy (FTIR) Determination by:

analyzing lyophilized composite nanoparticles with a FTIR, with a wavelength range of 4000 to 400 cm⁻¹ for a total of 32 scans, and a resolution of 4 cm⁻¹.

In some embodiments, evaluation of stability includes:

### (I) Assessing Storage Stability:

storing freshly prepared nanoparticles at 4° C. for 7, 15, and 30 days, evaluating a mean particle size and ζ potential of the nanoparticles afterward;

### J) Assessing pH and Ionic Stability:

adjusting a freshly prepared nanoparticle dispersion to pH values of 2.0, 4.0, 6.0, 8.0, and 10.0 using HCl or NaOH solutions for pH stability testing; mixing 2 mL of the freshly prepared nanoparticle dispersion with 2 mL of different concentrations of NaCl solution to prepare a series of nanoparticle dispersions with varying NaCl concentrations for ionic stability testing; and storing all samples at 4° C. for 24 hours before measuring the size, polydispersity index (PDI), and ζ potential of the nanoparticles;

### (K) Evaluating Thermal Stability:

heating the freshly prepared nanoparticle dispersion at 80° C. for 120 minutes, performing measurement every 30 minutes; cooling the nanoparticles to 25° C. after the heating and then measuring particle size, PDI, and surface charge; and

### (L) Assessing Photostability:

exposing a sample to ultraviolet (UV) light for various irradiation durations: 30 minutes, 60 minutes, 90 minutes, 120 minutes, 150 minutes, 180 minutes, 210 minutes, 240 minutes, 270 minutes, 300 minutes, and 12 hours; and determining retention of phytol content using GC-MS.

In some embodiments, a method of in vitro gastrointestinal digestion includes:
conducting in vitro digestion using a simulated gastric fluid (SGF) and a simulated intestinal fluid (SIF) on both unencapsulated phytol and phytol-loaded nanoparticles, including adjusting a pH of 10 mL of the sample to 2.5 using 5M HCl before SGF treatment; mixing the sample with a SGF containing 3.2 mg/mL pepsin and 2.0 mg/mL NaCl; transferring a resulting mixture quickly to a constant temperature shaker, gently stirring at 37° C. at 120 rpm for 90 minutes; and extracting the sample every 30 minutes during a SGF digestion;
after SGF digestion, digesting the sample by SIF, comprising adjusting the pH of the sample to 6.5 using 1M NaHCO₃ to inactivate pepsin; adding 10 mL of an SGF-digested sample to 10 mL of the SIF composed of 10 mg/mL bile salts, 3.20 mg/mL trypsin, 6.80 mg/mL K₂HO₄, and 8.80 mg/mL NaCl; re-adjusting the pH to 7.4 using 1M NaOH before starting SIF digestion; placing the mixture in a 37° C. constant temperature shaker, gently stirring at 120 rpm for 120 minutes; and calculating the retention rate of phytol obtained from the SGF and SIF digestions.

In some embodiments, a method for evaluating antioxidant activity of the present disclosure includes:

### (I) Assessing Scavenging Activity of Hydroxyl Radicals by:

mixing 1.00 mL of the nanoliposomes with 1.00 mL of 9.00 mmol/L salicylic acid ethanol solution, 1.00 mL of 9.00 mmol/L FeCl₂, and 1.00 mL of 8.80 mmol/L H₂O₂; incubating a resulting mixture in the dark at 37° C. for 30 minutes; measuring an absorbance of a reaction mixture at 536 nm using a multifunctional microplate reader and calculating a hydroxyl radical scavenging rate;

### (J) Assessing ABTS⁺ Scavenging Activity by:

storing a mixed solution of 7.00 mM 2,2′-azinobis(3-ethylbenzothiazoline-6-sulfonic acid) and 2.45 mM potassium persulfate in the dark at room temperature for 24 hours; diluting a working solution of 2,2′-azinobis(3-ethylbenzothiazoline-6-sulfonic acid) 50 times to achieve an absorbance of 0.70±0.02 at 734 nm; mixing 1.00 mL of the diluted solution with 19 mL of the working solution of 2,2′-azinobis(3-ethylbenzothiazoline-6-sulfonic acid) and performing incubation at room temperature for 1 hour; and measuring an absorbance at 734 nm and calculating the ABTS radical scavenging activity of the sample;

### (K) Culturing Cells by

culturing HepG2 cells in DMEM and maintaining the HepG2 cells in a cell incubator at 37° C. with a 5% CO₂ atmosphere;

### (L) Assessing Cell Viability by:

determining the cell viability using a CCK-8 assay.

### (M) Determining Tert-Butyl Hydrogen Peroxide (TBHP)-Induced Oxidative Damage Concentration

seeding 1×10⁵ cells per well of digested HepG2 cells into a 96-well cell culture plate; diluting TBHP in a serum-free Dulbecco's modified Eagle's medium (DMEM) to concentrations of 5.00, 10.00, 25.00, 50.00, 75.00, and 100.00 mg/mL; incubating the cells for 24 hours, then introducing into the plate and incubating the cells for another 24 hours, followed by determining cell viability using the CCK-8 assay;

### (N) Determining Phytol Concentration by:

seeding 1×10⁵ cells per well of the digested HepG2 cells into a 96-well cell culture plate and incubating in a cell incubator; after 24 hours of incubation, discarding a culture medium and adding different concentrations of phytol working solution to the 96-well cell culture plate; diluting a phytol stock solution in serum-free DMEM to 100.00, 200.00, 400.00, and 600.00 mg/mL, and adding the phytol working solution to the 96-well cell culture plate; continuing to incubate the cells for 24 hours, followed by determining HepG2 cell viability using the CCK-8 assay;

### (O) Establishing a Cellular Antioxidant Model by:

culturing the digested HepG2 cells for 24 hours and then adding 100.00 mg/mL concentrations of PHY, P-ZF, P-NL, and P-NL-ZF to the plate for another 24 hours of incubation; removing previous culture medium and adding 5.00 mg/mL TBHP working solution to a same plate; measuring a cell survival rate, an intracellular reactive oxygen species (ROS) level, malondialdehyde (MDA) content, and superoxide dismutase (SOD) activity.

### (P) Measuring ROS, SOD Activity, and MDA Content in the Cells

placing the digested HepG2 cells in a culture dish and performing a treatment as in step (O); adding 10.00 μM 2′,7′-dichlorodihydrofluorescein diacetate (DCFH-DA) and incubating for 0.5 hours, followed by washing with PBS (pH 7.4); allowing intracellular esterase to cleave the DCFH-DA, which is then oxidized to highly fluorescent dichlorofluorescein in the presence of ROS; measuring a fluorescence intensity of the 96-well plate using a multifunctional microplate reader at an excitation wavelength of 485 nm and an emission wavelength of 528 nm, expressing the intracellular ROS level as a percentage of the negative control group;
assessing the SOD activity and the MDA content by culturing the digested HepG2 cells for 24 hours; after the treatment as in step (O), adding 300 μL of lysis buffer and incubating on ice for 30 minutes; centrifuging at 11,000×g for 5 minutes at 4° C. and collecting a resulting supernatant; and quantifying the SOD activity and the MDA content according to instructions of SOD and MDA assay kits; and

### (Q) Observing ROS Production Using a Microscope by:

seeding the digested cells at a density of 3×10⁵ cells per well into a 6-well plate and incubating for 24 hours; after incubation, aspirating a culture medium and continuing with the grouping and treatment as in step (0); after 24 hours, replacing an original solution with 25 μM DCFH-DA solution and incubating for 60 minutes; washing the cells twice with PBS to remove unabsorbed probes; observing a fluorescence generated within the cells by placing the 6-well plate under a fluorescence microscope equipped with an FITC fluorescence channel.

Compared with the prior art, the technical solution of the present disclosure have the following advantages and effects: zein and fucoidan are used as composite biopolymers to modify nanoliposomes loaded with phytol, thereby enhancing the performance of phytol nanoparticles. The present disclosure involves the preparation of nanoliposomes conjugated with zein and fucoidan for the encapsulation of phytol, aimed at improving its water solubility and chemical stability. The encapsulation efficiency of phytol in various nanoparticles is tested, and the effects of preparation conditions on the morphology, mean particle size, polydispersity index (PDI), zeta potential, and encapsulation efficiency of the nanoparticles are evaluated using FTIR, zeta potential analyzer, transmission electron microscopy (TEM), and GC-MS. Furthermore, the biocompatibility of the prepared nanoparticles is assessed through in vitro simulated digestion experiments, while their antioxidant activity is evaluated using in vitro chemical and cellular antioxidant assays. The technical solution of the present disclosure contributes to the design of novel nano-delivery systems for the encapsulation, protection, and release of phytol and similar bioactive substances.

### BRIEF DESCRIPTION OF THE DRAWINGS

To illustrate the technical solutions of the embodiments of the present disclosure, a brief description to the accompanying drawings used is provided below.

FIG. 1 is a schematic flow chart of the preparation process of the examples of the present disclosure.

FIG. 2A is a representative TEM image of P-NL-ZF-S with an enlarged image in an example of the present disclosure.

FIG. 2B is a representative TEM image of another P-NL-ZF-S with an enlarged image in an example of the present disclosure.

FIG. 2C is a representative TEM image of P-NL-ZF-HPH with an enlarged image in an example of the present disclosure.

FIG. 2D is a representative TEM image of another P-NL-ZF-HPH with an enlarged image in an example of the present disclosure.

FIG. 2E is a representative TEM image of P-NL-ZF-S-HPH with an enlarged image in an example of the present disclosure.

FIG. 2F is a representative TEM image of another P-NL-ZF-S-HPH with an enlarged image in an example of the present disclosure.

FIG. 3A is a representative FTIR plot of fucoidan, zein, and phytol.

FIG. 3B is a representative FTIR plot of blank NL, blank ZF, blank NL-ZF, P-NL, P-ZF, and P-NL-ZF.

FIG. 4A is a graph showing the particle size variation curve of different nanoparticles stored at 4° C. for 30 days according to the present disclosure.

FIG. 4B is a graph showing the polydispersity index variation curve of different nanoparticles stored at 4° C. for 30 days according to the present disclosure.

FIG. 4C is a graph showing the zeta potential variation curve of different nanoparticles stored at 4° C. for 30 days according to the present disclosure.

FIG. 4D is a graph showing the PHY release rate variation curve of different nanoparticles stored at 4° C. for 30 days according to the present disclosure.

FIG. 5A is a bar plot illustrating the effect of different pH values on particle size in an example of the present disclosure.

FIG. 5B is a curve plot illustrating the effect of different pH values on zeta potential in an example of the present disclosure.

FIG. 5C is a bar plot illustrating the effect of NaCl concentration (0-80 mM) on particle size.

FIG. 5D is a curve plot illustrating the effect of NaCl concentration (0-80 mM) on zeta potential in an example of the present disclosure.

FIG. 5E is a bar plot illustrating the effect of thermal conditions on particle size in an example of the present disclosure.

FIG. 5F is a curve plot illustrating the effect of thermal conditions on zeta potential in an example of the present disclosure.

FIG. 6A is a curve plot illustrating the retention rates of nanoparticles (P-ZF, P-NL, and P-NL-ZF) and free phytol in ethanol under UV conditions in an example of the present disclosure.

FIG. 6B is a curve plot illustrating the retention rates of nanoparticles (P-ZF, P-NL, and P-NL-ZF) and free phytol in ethanol after 90 minutes of simulated gastric fluid (SGF) digestion and 150 minutes of simulated intestinal fluid (SIF) digestion in an example of the present disclosure.

FIG. 7A is a bar plot of the ABTS⁺ radical scavenging activity according to the present disclosure, with different letters indicating significant differences (p<0.05).

FIG. 7B is a bar plot of hydroxyl radical scavenging activity according to the present disclosure, with different letters indicating significant differences (p<0.05).

FIG. 8A is a bar plot illustrating the cell viability of HepG2 cells treated with different concentrations (PHY 50-400 μg/mL) of free phytol, P-ZF, P-NL, and P-NL-ZF according to the present disclosure, with different letters indicating significant differences (p<0.05).

FIG. 8B is a bar plot illustrating the effect of different concentrations of TBHP (5-100 g/mL) on the cell viability of HepG2 cells according to the present disclosure, with different letters indicating significant differences (p<0.05).

FIG. 8C is a bar plot illustrating the effect of free phytol, P-ZF, P-NL, and P-NL-ZF on the ROS content in HepG2 cells treated with TBHP (10 μg/mL) according to the present disclosure, with different letters indicating significant differences (Pp<0.05).

FIG. 8D is a bar plot illustrating the effect of free phytol, P-ZF, P-NL, and P-NL-ZF on the MDA content in HepG2 cells treated with TBHP (10 μg/mL) according to the present disclosure, with different letters indicating significant differences (p<0.05).

FIG. 8E is a bar plot illustrating the effect of free phytol, P-ZF, P-NL, and P-NL-ZF on the SOD activity in HepG2 cells treated with TBHP (10 μg/mL) according to the present disclosure, with different letters indicating significant differences (p<0.05).

### DETAILED DESCRIPTION OF THE EMBODIMENTS

The following detailed explanation of the present disclosure is provided in conjunction with the accompanying figures and through examples. The examples below serve as an explanation of the present disclosure, which is unlimited to the following examples.

### Example 1

### 1. Materials

Lecithin (derived from egg yolk, molecular weight: 758.06 kDa, purity: >80%), phytol (purity: 95%), zein (purity: 92%), cholesterol (purity: 99%), anhydrous potassium dihydrogen phosphate (purity: 99%), and n-hexane (chromatographic purity) were purchased from Shanghai Acmec Biochemical Co., Ltd. (Shanghai, China); fucoidan, with an average molecular weight (Mw) of 120 kDa (extracted from kelp (Laminaria japonica)), was obtained from Shandong Jiejing Group Co., Ltd. (Rizhao, China); pepsin (1:3000), trypsin (1:250), bile salts, and phosphate-buffered saline (PBS) were acquired from Solarbio Science & Technology Co., Ltd. (Beijing, China). DMEM, penicillin-streptomycin mixed solution (10,000 units/mL and 10 mg/mL, respectively), trypsin-EDTA, fetal bovine serum (FBS), and human liver cancer HepG2 cell line were sourced from Shanghai Fuheng Biotechnology Co., Ltd. (Shanghai, China); cell counting kit-8 (CCK-8), superoxide dismutase (SOD), malondialdehyde (MDA), and reactive oxygen species (ROS) detection kits were obtained from Byotime Biotechnology (Shanghai) Co., Ltd.; TBHP was purchased from Alfa Aesar (China) Chemical Co., Ltd.; and MDA protein detection kits were sourced from Nanjing Jiancheng Bioengineering Institute (Nanjing, China). Ethanol, hydrochloric acid, and sodium hydroxide of analytical grade were acquired from Xilong Scientific Co., Ltd. (Guangdong, China).

### 2. Methods

### 2.1 Preparation of Zein-Fucoidan Nanoparticles

Firstly, zein was dissolved in a 75% ethanol solution to prepare a zein stock solution (1% w/v). A solution of fucoidan (200 mg) was prepared by dissolving it in 40 mL of high-purity water, and the pH was adjusted to 3.5. The zein stock solution (10 mL) was mixed with the fucoidan solution and stirred vigorously at a speed of 1000 g for 60 minutes. Subsequently, ethanol was removed from the mixture using a vacuum rotary evaporator, and the resulting solution was diluted to 50 mL with water at a pH of 3.5. Finally, the solution was centrifuged (1500 g, 10 min) to remove insoluble materials, and the collected colloidal particle dispersion was obtained.

### 2.2 Preparation of Nanoliposomes

Firstly, lecithin, cholesterol, and phytol were dissolved in 10 mL of ethanol and stirred to ensure complete dissolution. Secondly, ethanol was removed using a rotary evaporator at 45° C. and a rotation speed of 70 rpm to form a thin film on the container's surface. Thirdly, the film was dried overnight in an oven at 30° C. to completely remove ethanol. Fourthly, the film was hydrated with 20 mL of ultrapure water and stirred continuously at 700 rpm and 50° C. for 1 hour. After hydration, ultrasonic treatment was performed for 15 minutes using a cell disruptor (JY92-IIN, Scientz Biotechnology, Ningbo, China) in an ice bath (with 5 seconds on and 5 seconds off cycles) to obtain a uniform liposome solution. Finally, the nanoliposomes were stored at 4° C. for subsequent experiments.

### 2.3 Preparation of Biopolymer-Conjugated Nanoliposomes

Zein-fucoidan nanoparticles and nanoliposomes were prepared in a molar ratio of 1:20 (zein: cholesterol) to create biopolymer-conjugated nanoliposomes. Three different methods were employed: magnetic stirring at 700 rpm for 3 hours, high-pressure homogenization (AH100D, ATS ENGINEERING INC., Shanghai, China) at 80 bar for 3 cycles, and a combined method of the first two approaches. Subsequently, all nanoparticles were stored in a 4° C. environment until needed.

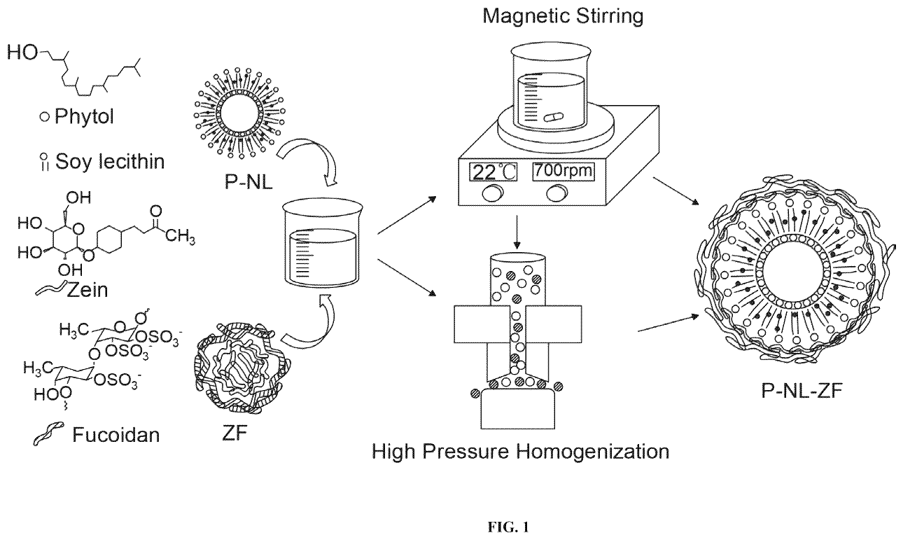

### 2.4 Structure and Preparation Process of P-NL-ZF, as FIG. 1 Visually Illustrated.

The obtained formulations were designated as follows: P-ZF, P-NL, P-NL-ZF-S, P-NL-ZF-HPH, and P-NL-ZF-S-HPH. Herein, “P” represents phytol, “Z” represents zein, “F” represents fucoidan, “NL” represents nanoliposomes, “S” represents magnetic stirring, and “HPH” represents high-pressure homogenization.

### 2.5 Characterization of Nanoparticles

### 2.5.1 Measurement of Particle Size and Zeta Potential

The mean particle size (MPS), polydispersity index (PDI), and zeta potential ((potential) of the composite particles were analyzed using a zetasizer (Nano ZS90, Malvern Instruments Co., Malvern, UK) through dynamic light scattering (DLS) and electrophoretic mobility techniques. To mitigate multiple scattering effects, the particle dispersion was appropriately diluted 10 times with distilled water.

### 2.5.2 Encapsulation Efficiency (EE %) Analysis

The suspension of nanoparticles was centrifuged at 24,200 g for 22 minutes at 4° C. A resulting supernatant was then separated, and the solid components were washed twice with PBS (pH 7.4). Phytol was extracted from the nanoparticles and the supernatant using n-hexane, followed by ultrasonic treatment at 30° C. for 20 minutes. Quantitative analysis was conducted using GC-MS (Agilent 7890A/5975C, Agilent Technologies, Palo Alto, USA) to calculate the encapsulation efficiency. The calculation formula is as follows:

$$EE ( \% ) = \frac{Encapsulated PHY}{( Encapsulated PHY + Free PHY )} \times 100$$

where EE(%) represents encapsulation efficiency; Encapsulated PHY represents encapsulated phytol; and Free PHY represents unencapsulated phytol.

### 2.5.3 GC-MS Assay

The phytol content was analyzed using a 7890A-5975C GC-MS system. The chromatographic column used was Agilent J&W DB-5 ms (30 m×0.25 mm×0.25 μm), with high-purity helium as the carrier gas at a flow rate of 1.0 mL/min. The injector temperature was set at 250° C., with a sample volume of 4.0 μL, employing a splitless injection method. The temperature program was set to hold at 100° C. for 2 minutes, then increased to 250° C. at a rate of 5° C./min, and maintained for 32 minutes. The ion source temperature of the mass spectrometer was maintained at 230° C., with the transfer line temperature set to 280° C. The scanning mass range was set from 35 to 600 amu, with an energy of 70 eV, and a solvent delay time of 4 minutes. The obtained chromatographic ion fragmentation patterns were cross-referenced with the ion fragments in the NIST 2005 database (National Institute of Standards and Technology, USA) for the qualitative identification of the target compound.

For the quantification of phytol, 0.0100 g of phytol was weighed, dissolved in n-hexane, and diluted to a final volume of 10 mL in a brown volumetric flask. Standard solutions were prepared at concentrations of 1.00 mg/mL, 0.40 mg/mL, 0.20 mg/mL, 0.10 mg/mL, and 0.05 mg/mL using n-hexane, filtered through a 0.45 μm nylon membrane, and analyzed using GC-MS. The linear regression equation obtained from the phytol standard sample was: y=2E+09X+9E+06 (R²=0.9983). This equation was used to calculate the encapsulation rate of phytol.

### 2.5.4 Transmission Electron Microscopy (TEM)

High-resolution TEM (JEM-2100, Nippon Electronic Co.) was employed to observe the morphology of vesicles in the optimized liposome batches. Each sample was diluted with distilled water after preparation and carefully placed as single droplets on carbon-coated copper grids, followed by drying. To enhance the visibility of lipid components, the grids were inverted onto a 2% phosphotungstic acid solution for negative staining.

### 2.5.5 Fourier Transform Infrared Spectroscopy (FTIR)

Fourier transform infrared spectroscopy was performed using a Nicolet iS10 spectrometer (Thermo Fisher Scientific, Massachusetts, USA) to analyze the lyophilized composite nanoparticles. The scanning wavelength range was set from 4000 to 400 cm⁻¹, with a total of 32 scans and a resolution of 4 cm⁻¹.

### 2.5.6 Stability Assessment

### 2.5.6.1 Storage Stability

Freshly prepared nanoparticles (P-ZF, P-NL, and P-NL-ZF) were stored at 4° C. for 7, 15, and 30 days, respectively. Subsequently, the mean particle size (MPS) and zeta potential of the nanoparticle samples were evaluated. The determination of phytol content followed the method described in section 2.5.2, and the release rate calculation formula is as follows:

$$Release Rate ( \% ) = ( \frac{( {C}_{0} - C )}{{C}_{0}} ) \times 100$$

where C₀ and C are the initial and remaining phytol amounts in the particles, respectively.

### 2.5.6.2 pH and Ionic Stability

For pH stability testing, fresh nanoparticle dispersion was adjusted to pH values of 2.0, 4.0, 6.0, 8.0, and 10.0 using HCl or NaOH solutions. For ionic stability testing, 2 mL of freshly prepared nanoparticle dispersion (P-ZF, P-NL, and P-NL-ZF) was mixed with 2 mL of NaCl solutions at different concentrations (0, 20, 40, 60, and 80 mM) to prepare a series of nanoparticle dispersions with varying NaCl concentrations. All samples were stored at 4° C. for 24 hours, and the particle size, PDI, and zeta potential were then measured.

### 2.5.6.3 Thermal Stability

Freshly prepared nanoparticle dispersions (P-ZF, P-NL, and P-NL-ZF) were heated at 80° C. for 120 minutes, with measurements taken every 30 minutes. After heated, the nanoparticles were cooled down to 25° C., and their size, polydispersity index (PDI), and surface charge were measured.

### 2.5.6.4 Photostability

To assess the photostability of phytol loaded in nanoparticles (P-ZF, P-NL, and P-NL-ZF) as well as free phytol dissolved in ethanol, samples were irradiated under UV light for various durations: 30 minutes, 60 minutes, 90 minutes, 120 minutes, 150 minutes, 180 minutes, 210 minutes, 240 minutes, 270 minutes, 300 minutes and 12 hours. The content of retained phytol was determined using GC-MS as described in Section 2.5.2. The calculation formula for retention rate is given as follows:

$$Retention rate ( \% ) = \frac{C}{{C}_{0}} \times 100$$

where C₀ and C represent the initial content of phytol in the particles and the retained amount, respectively.

### 2.5.6.5 In Vitro Gastrointestinal Digestion

In vitro digestion experiments were conducted using simulated gastric fluid (SGF) and simulated intestinal fluid (SIF) on both unencapsulated phytol and phytol-loaded nanoparticles (P-ZF, P-NL, and P-NL-ZF). Prior to the SGF treatment, a 10 mL sample was taken and its pH was adjusted to 2.5 using 5 M HCl. Following this, a mixture containing pepsin (3.2 mg/mL) and NaCl (2.0 mg/mL) was added to create the SGF. The resulting mixture was then quickly transferred to a thermostatic shaker and gently stirred at 120 rpm for 90 minutes at a temperature of 37° C. Samples were extracted at intervals of 30 minutes.

After digestion in SGF, the samples were subjected to SIF digestion. The pH was adjusted to 6.5 using 1 M NaHCO₃ to inactivate pepsin under this specific pH condition. Subsequently, 10 mL of the SGF-digested sample were added to a simulated intestinal fluid composed of 10 mg/mL bile salts, 3.20 mg/mL trypsin, 6.80 mg/mL K₂HPO₄, and 8.80 mg/mL NaCl (totaling 10 mL). Before initiation of SIF digestion, the pH was readjusted to 7.4 using 1 M NaOH. The resulting mixture was then placed in a 37° C. thermostatic oscillator and gently shaken at 120 rpm for 120 minutes. As described in Section 2.5.6.3, the samples obtained from SGF and SIF digestions were utilized for the study of phytol retention rates.

### 2.6 Measurement of Antioxidant Activity

### 2.6.1 Hydroxyl Radical Scavenging Activity

A sample of the nanoliposome (1.00 mL) was mixed with an ethanol solution of salicylic acid (1.00 mL, 9.00 mmol/L), FeCl₂ (1.00 mL, 9.00 mmol/L), and H₂O₂(1.00 mL, 8.80 mmol/L) and incubated in the dark at 37° C. for 30 minutes. The absorbance of the reaction mixture was measured at 536 nm using a multifunctional microplate reader (Varioskan Flash, Thermo Fisher Scientific, Massachusetts, USA). The hydroxyl radical scavenging rate was calculated using the following formula:

$$The hydroylscavenging ( \% ) = [ 1 - \frac{( {A}_{1} - {A}_{2} )}{{A}_{0}} ] \times 100$$

where A₁ is the absorbance of the blank sample; A₀ is the absorbance of the control sample; and A₂ is the absorbance of the sample without the ethanol solution of salicylic acid.

### 2.6.2 ABTS⁺ Scavenging Activity

Prior to use, a mixed solution of 2,2′-azino-bis(3-ethylbenzothiazoline-6-sulfonic acid) (ABTS) (7.00 mM) and potassium persulfate (2.45 mM) was stored in the dark at room temperature for 24 hours. Subsequently, the ABTS working solution was diluted approximately 50 times to achieve an absorbance value of 0.70±0.02 at a wavelength of 734 nm. Then, 1.00 mL of the diluted solution was mixed with 19 mL of the ABTS working solution and incubated at room temperature for 1 hour. The absorbance at 734 nm was measured. The ABTS⁺ radical scavenging activity of the sample was calculated using the formula:

$$ABTS scavenging activity ( \% ) = ( \frac{{A}_{0} - {A}_{1}}{{A}_{0}} ) \times 100 \%$$

where A₀ is the absorbance of the blank solution, and A₁ is the absorbance of the sample solution.

### 2.6.3 Cell Culture

HepG2 cells were cultured in DMEM, supplemented with 10% fetal bovine serum (FBS) and 1% penicillin-streptomycin mixed solution (containing 10,000 units/mL of penicillin and 10 mg/mL of streptomycin). The cells were maintained in a cell culture incubator at a temperature of 37° C. and a carbon dioxide concentration of 5%.

### 2.6.4 Cell Viability

Cell viability was assessed using the CCK-8 assay. After being cultured in the incubator for 24 hours, the cells were seeded into a 96-well plate at a density of 1×10⁵ cells per well. Subsequently, the cells were exposed to different concentrations (0, 100.00, 200.00, 400.00, and 600.00 mg/mL) of nanoparticle dispersions (P-ZF, P-NL, and P-NL-ZF). After 24 hours, the culture medium was removed from each well, and the treated cells were washed twice with PBS. In each well, 90 μL of serum-free DMEM and 10 μL of CCK-8 solution were added. The cells were then incubated at 37° C. for an additional 0.5 hours. The absorbance was measured at a wavelength of 450 nm using a multifunctional microplate reader (Varioskan Flash, Thermo Fisher Scientific). The calculation formula for cell viability is as follows:

$$Cell Viability ( \% ) = \frac{{A}_{1} - {A}_{0}}{{A}_{2} - {A}_{0}} \times 100$$

where A₀ is the absorbance measured at 450 nm for the blank group sample, A₁ is the absorbance measured at 450 nm for the treatment group sample, and A₂ is the absorbance measured at 450 nm for the control group sample.

### 2.6.5 Determination of TBHP-Induced Oxidative Damage Concentration

HepG2 cells, after digestion, were seeded at a density of 1×10⁵ cells/well in a 96-well cell culture plate and were incubated in a cell culture incubator. TBHP was diluted in serum-free DMEM to concentrations of 5.00, 10.00, 25.00, 50.00, 75.00, and 100.00 mg/mL. After 24 hours of incubation, the culture medium was removed, and the working solutions of TBHP at different concentrations were added to the wells, followed by an additional 24 hours of incubation. After the incubation period, the vitality of the HepG2 cells was assessed using the CCK-8 method, as detailed in section 2.6.4.

### 2.6.6 Determination of Phytol Concentration

HepG2 cells, after digestion, were seeded at a density of 1×10⁵ cells/well in a 96-well cell culture plate and were placed in a cell incubator. After 24 hours of incubation, the culture medium was discarded, and different concentrations of phytol working solution were added to the wells. The phytol stock solution was diluted in serum-free DMEM to concentrations of 100.00, 200.00, 400.00, and 600.00 mg/mL, and these dilutions were introduced into the wells. Following the protocol described in section 2.6.4, the cells were incubated for an additional 24 hours before the vitality of the HepG2 cells was measured using the CCK-8 assay.

### 2.6.7 Establishment of Cellular Antioxidant Model

HepG2 cells, after digestion, were seeded at a density of 1×10⁵ cells/well in a 96-well cell culture plate and were placed in a cell culture incubator. After 24 hours of incubation, the initial culture medium was discarded, and solutions of PHY, P-ZF, P-NL, and P-NL-ZF at a concentration of 100.00 mg/mL were added to the wells for an additional 24 hours of incubation. Subsequently, the previous culture medium was removed, and a working solution of TBHP at a concentration of 5.00 mg/mL was added to the same wells. Following these treatments, the cell viability, intracellular ROS levels, MDA content, and SOD activity were determined. These analyses aid in understanding the effects of interventions in alleviating oxidative damage in HepG2 cells.

### 2.6.8 Measurement of ROS, SOD Activity, and MDA Content in Cells

Initially, HepG2 cells, after digestion, were seeded at a density of 1×10⁵ cells/well in a 96-well cell culture plate with a black outer surface and a transparent bottom, and were subjected to the experimental treatments described in section 2.6.7. Following this, 10.00 μM DCFH-DA was added and incubated for 0.5 hours, followed by washing with PBS. Intracellular esterases cleave DCFH-DA, resulting in its oxidation into highly fluorescent dichlorofluorescein (DCF) in the presence of ROS. The fluorescence intensity of the 96-well plate was measured using a multifunctional microplate reader at an excitation wavelength of 485 nm and an emission wavelength of 528 nm. The intracellular ROS levels of the experimental groups were expressed as a percentage of the negative control group, calculated using the following formula:

$$ROS level ( \% ) = \frac{F}{{F}_{0}} \times 100$$

where F represents fluorescence intensity of the damage model group or drug intervention group, and F₀ represents fluorescence intensity of the blank control group.

To evaluate SOD activity and MDA content, digested HepG2 cells (6×10⁵) were transferred to 6-well cell culture plates and incubated for 24 hours. After various treatments (following the same experimental protocol detailed in section 2.6.7), 300 μL of lysis buffer was added to each well and incubated on ice for 30 minutes. The samples were then centrifuged at 11000×g for 5 minutes at 4° C., and a resulting supernatant was collected. The quantification of SOD activity and MDA content was performed according to the instructions provided in the SOD and MDA assay kits.

### 2.6.9 Observation of ROS Production by Microscopy

Cells that had been digested were seeded in a 6-well plate at a density of 3×10⁵ cells per well and incubated for 24 hours. At the end of the incubation period, the culture medium was removed, and treatments were continued according to the grouping described in section 2.6.7. Twenty-four hours later, the culture medium was replaced with DCFH-DA solution (25 μM) and incubated for 60 minutes. Subsequently, the cells were washed twice with PBS to remove any unabsorbed probe. To observe the intracellular fluorescence, the 6-well plate was placed under a fluorescence microscope (DM6B, Leica, Heidelberg, Germany) equipped with a FITC fluorescence channel to visualize the production of intracellular fluorescence.

### 3. Results and Discussion

### 3.1 Characterization of Nanoparticles

TABLE 1
Average diameter, polydispersity index, zeta potential and encapsulation
efficiency of the nanoparticles loaded with PYT.

| | Average | Polydispersity | Zeta | Encapsulation |
|---|---|---|---|---|
| Samples | diameter (nm) | Index | potential (mV) | efficiency % |
| BNL | 86.30 ± 0.51c | 0.341 ± 0.05a | −63.10 ± 0.85c | — |
| P-NL | 97.88 ± 1.30c | 0.250 ± 0.07a | −65.87 ± 3.14c | 85.85 ± 0.15a |
| BZF | 139.50 ± 0.50a | 0.205 ± 0.01b | −54.40 ± 0.51b | — |
| P-ZF | 122.23 ± 2.98b | 0.220 ± 0.01b | −33.03 ± 1.37a | 67.49 ± 0.11b |
| P-NL-ZF-S | 151.22 ± 13.39a | 0.388 ± 0.06a | −78.02 ± 1.18e | 72.14 ± 0.23e |
| P-NL-ZF-HPH | 144.01 ± 5.39a | 0.395 ± 0.05a | −70.90 ± 1.62e | 66.31 ± 0.28c |
| P-NL-ZF-S-HPH | 142.14 ± 8.66a | 0.235 ± 0.01ab | −74.93 ± 1.10d | 76.19 ± 0.03d |

The data in Table 1 are expressed as mean standard deviation. Means with different letters in the same column indicates significant differences (P<0.05). PHY represents phytol; P-ZF represents fucoidan composite nanoparticles loaded with PHY; P-NL represents nanoliposomes loaded with PHY; BZF represents blank zein-fucoidan composite nanoparticles; P-NL-ZF-S represents PHY-loaded nanoliposomes to which ZF is conjugated through magnetic stirring; P-NL-ZF-HPH represents PHY-loaded nanoliposomes to which ZY is conjugated through high-pressure homogenization; P-NL-ZF-S-HPH represents PHY-loaded nanoliposomes to which ZF is conjugated through both magnetic stirring and high-pressure homogenization.

Table 1 lists the mean particle size, PDI, zeta potential, and encapsulation efficiency (EE) of various phytol-loaded nanoparticles. By comparing the particle sizes of BNL and P-NL, along with GC-MS analysis, it was confirmed that PHY was encapsulated within the nanoliposomes. Due to its hydrophobic nature, PHY was found to reside in the lipid phase of the liposomes through hydrogen bonding interactions. Both P-NL and P-ZF exhibited good dispersibility (PDI<0.300) with particle sizes <130.00 nm and zeta potentials of negative values (−65.87 mV and −33.02 mV, respectively).

The introduction of the biopolymer (ZF) to P-NL resulted in an increase in particle size: the particle size of P-NL-ZF-S was 151.22 nm, P-NL-ZF-HPH was 144.01 nm, and P-NL-ZF-S-HPH was 142.14 nm, representing increases of 54.50%, 47.13%, and 45.22%, respectively, compared to that of P-NL. The surface charge transitioned to a larger absolute value, from −65.87 mV to −78.02 mV, indicating that the increase in potential enhanced the repulsive forces between the vesicles, which effectively prevented nanoparticle aggregation and maintained an acceptable PDI value. The change in zeta potential was associated with the absorption of negatively charged fucoidan (FU) by the positively charged zein outer layer, as well as the negative charges of the terminal groups on the phospholipids, strongly indicating the conjugation of ZF with P-NL. High-pressure homogenization (HPH) did not significantly alter the zeta potential of the biopolymer-conjugated nanoliposomes. However, HPH optimized the particle size and dispersibility of the conjugated nanoliposomes, reducing the mean particle size and PDI from 151.22 nm and 0.388 to 142.14 nm and 0.235, respectively. The observed trend of particle size reduction was attributed to the cavitation, friction, shear, and turbulence effects induced by HPH. This effect also enhanced the anti-flocculation and coagulation stability of the nanoparticles, as higher preparation pressures improved emulsification capacity, leading to a reduction in both particle size and PDI.

### 3.2 Microstructural and Morphological Analysis

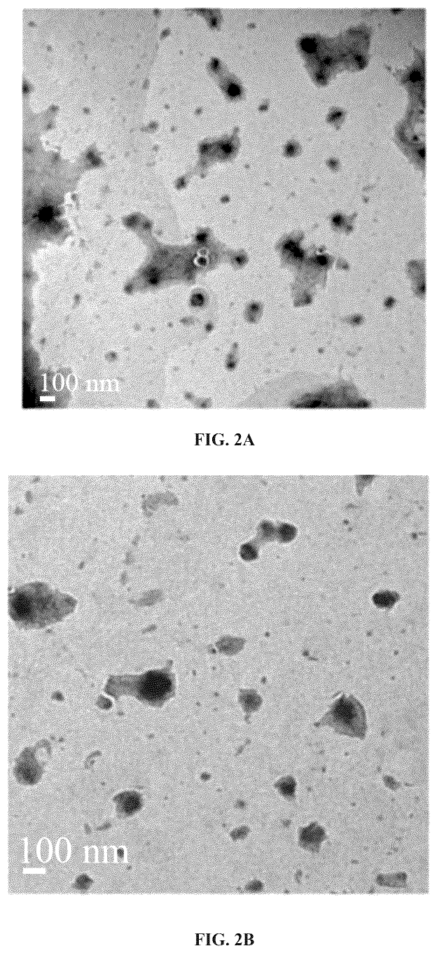

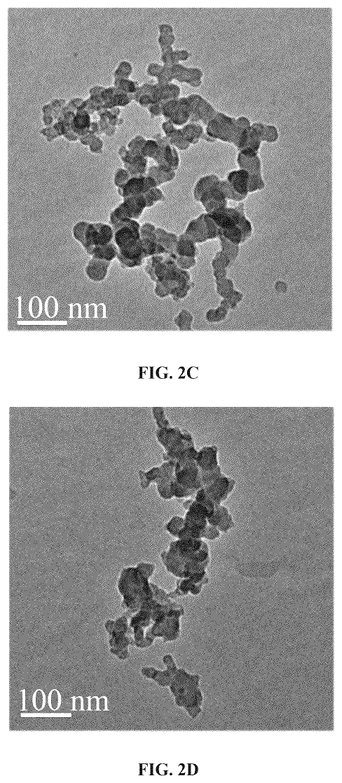

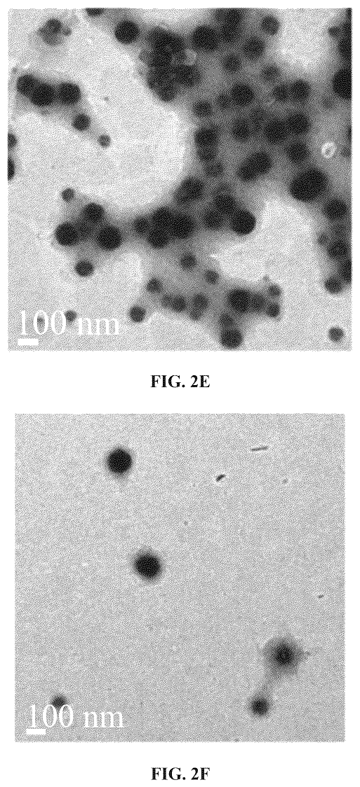

In order to evaluate the effect of HPH on the microstructure of nanoparticles, TEM was employed to observe the morphology of the nanoparticles. FIGS. 2A and 2B present the TEM images of P-NL-ZF-S at higher magnification, while FIGS. 2C and 2D depict the TEM images of P-NL-ZF-HPH, and FIGS. 2E and 2F show the TEM images of P-NL-ZF-S-HPH at higher magnification. Herein, P-NL-ZF-S represents PHY-loaded nanoliposomes to which ZF is conjugated through magnetic stirring; P-NL-ZF-HPH represents PHY-loaded nanoliposomes to which ZF is conjugated through high-pressure homogenization; and P-NL-ZF-S-HPH represents PHY-loaded nanoliposomes to which ZF is conjugated through both magnetic stirring and high-pressure homogenization.

The dark vesicles identifiable from FIGS. 2A to 2F correspond to the nanoliposomes. By comparison of the particle sizes obtained from dynamic light scattering (DLS) and TEM, the grayish-white edges observed on the surface of the nanoliposomes in the TEM images indicated the presence of a ZF coating, confirming the formation of the composite. The P-NL-ZF-S-HPH vesicles exhibited a uniform distribution and regular shape, contrasting sharply with the irregular shapes and vesicle aggregation observed in P-NL-ZF-HPH and P-NL-ZF-S. Additionally, numerous empty small vesicle fragments were found in P-NL-ZF-HPH, which aligned with previous data, indicating that P-NL-ZF-HPH and P-NL-ZF-S had poorer dispersibility, with the lowest encapsulation efficiency observed for P-NL-ZF-HPH.

### 3.3 Fourier Transform Infrared Spectroscopy Analysis of Nanoparticles

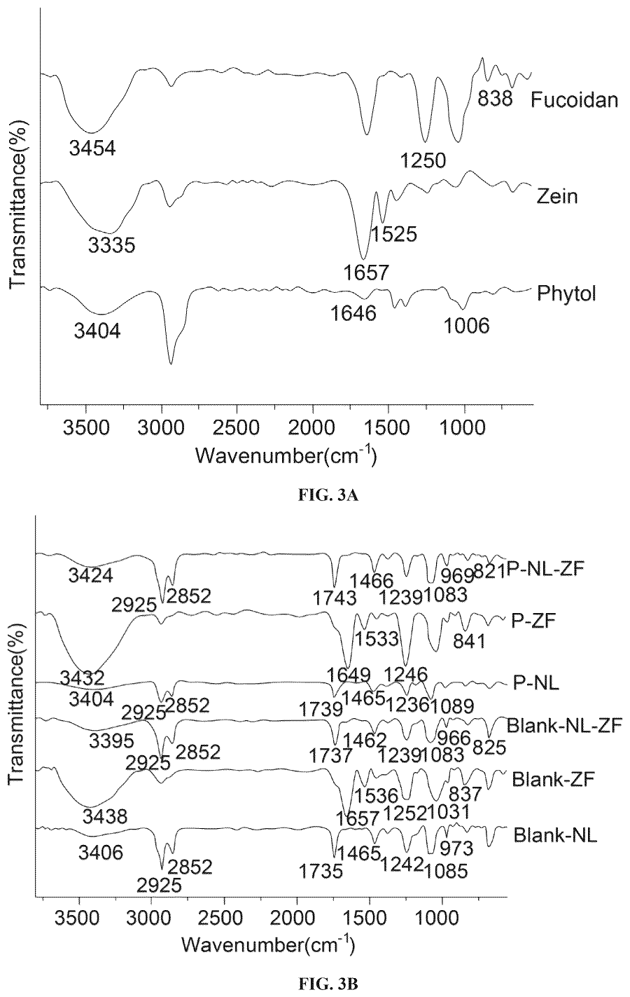

FIG. 3A displays the FTIR spectra of fucoidan, zein, and phytol; FIG. 3B presents the FTIR spectra of blank NL, blank ZF, blank NL-ZF, P-NL, P-ZF, and P-NL-ZF. In this context, P-ZF represents fucoidan composite nanoparticles loaded with PHY; P-NL represents nanoliposomes loaded with PHY; P-NL-ZF-S represents PHY-loaded nanoliposomes to which ZF is conjugated through magnetic stirring; P-NL-ZF-HPH represents PHY-loaded nanoliposomes to which ZF is conjugated through high-pressure homogenization; and P-NL-ZF-S-HPH represents PHY-loaded nanoliposomes to which ZF is conjugated through both magnetic stirring and high-pressure homogenization.

The FTIR spectra in FIG. 3A indicates the purified compounds (PHY, zein, fucoidan), while FIG. 3B shows the spectra of unencapsulated nanoparticles (blank-ZF, blank-NL, blank-ZF-NL) and PHY-loaded nanoparticles (P-ZF, P-NL, P-NL-ZF). These spectral data were further utilized to assess the interactions among zein, fucoidan, and nanoliposomes. The PHY exhibited characteristic peaks at specific wavelengths: 3404 cm⁻¹ (0-H stretching vibration), 1646 cm⁻¹ (C═C stretching vibration), and 1006 cm⁻¹ (C—OH stretching vibration). Notably, the fucoidan spectrum displayed significant absorption peaks at 1250 cm⁻¹ and 838 cm⁻¹, corresponding to S═O stretching vibrations and C—O—S bending vibrations, respectively, which were attributed to the presence of sulfate groups in fucoidan. The zein spectrum revealed characteristic absorption peaks at 1657 cm⁻¹ (amide I band, C—O stretching) and 1525 cm⁻¹ (amide II band, C—N stretching and bending vibrations of N—H groups). Furthermore, the fucoidan and zein exhibited typical 0-H stretching vibration bands at 3454 cm⁻¹ and 3335 cm⁻¹, respectively, which were consistent with previously reported results. Relative to pure zein, the O—H stretching vibration bands of blank-ZF and P-ZF were shifted to 3335 cm⁻¹, 3406 cm⁻¹, and 3432 cm⁻¹, respectively, while the amide II characteristic peak shifted from 1525 cm⁻¹ to 1536 cm⁻¹ (blank-ZF) and 1533 cm⁻¹ (P-ZF). These shifts indicate the formation of hydrogen bonds and electrostatic interactions between zein and fucoidan, leading to the formation of composite particles.

The spectrum of blank-NL displayed peaks distinct from those of the composite particles, where 1735 cm⁻¹ represents C═O symmetric stretching vibrations, 2925 cm⁻¹ and 2852 cm⁻¹ indicates symmetric and asymmetric CH₂ stretching vibrations of the acrylic chain, 1085 cm⁻¹ and 1242 cm⁻¹ relates to the symmetric and asymmetric stretching vibrations of PO²⁻, and 1465 cm⁻¹ corresponds to asymmetric stretching vibrations of CH₃. Upon the addition of PHY, no distinct characteristic absorption peaks of pure PHY were observed in P-ZF and P-NL, indicating effective encapsulation of PHY by ZF and NL. After ZF was conjugated to NL in blank-NL and P-NL, the positions of the symmetric and asymmetric C—H bonds in CH₂ stretching vibrations (2925 cm⁻¹ and 2852 cm⁻¹) remained unchanged. However, the carbonyl stretching vibrations (C═O) at 1735 cm⁻¹ (blank-NL) and 1739 cm⁻¹ (P-NL) shifted to 1737 cm⁻¹ (blank-NL-ZF) and 1743 cm⁻¹ (P-NL-ZF). Furthermore, significant electrostatic interactions between ZF and the nanoliposomes were indicated by the strengthening or development of hydrogen bonding in blank-NL-ZF and P-NL-ZF; the O—H band positions of NL shifted from 3406 cm⁻¹ (blank-NL) and 3404 cm⁻¹ (P-NL) to approximately 3495 cm⁻¹ (blank-NL-ZF) and 3424 cm⁻¹ (P-NL-ZF) in the composite particles; and the PO²⁻ peaks shifted (blank-NL: 1242 cm⁻¹ and 1085 cm⁻¹, blank-NL-ZF: 1239 cm⁻¹ and 1083 cm⁻¹). The amide II stretching band (1536 cm⁻¹) in blank-NL-ZF and P-NL-ZF nearly disappeared, which verified the presence of electrostatic and hydrogen bonding interactions between the alkyl chains of lecithin and zein. In addition, the blank-NL-ZF and P-NL-ZF exhibited characteristic peaks of Fu, specifically the C—O—S stretching vibrations at 825 cm⁻¹ and 821 cm⁻¹, confirming the successful conjugation of ZF on the surface of P-NL. A shift in the C—O—S stretching vibrations was also observed when fucoidan was conjugated to chitosan-coated nanoliposomes. In summary, the changes in the positions of the characteristic peaks indicated the presence of electrostatic interactions and hydrogen bonding between P-NL and ZF, ultimately resulting in the formation of P-NL-ZF.

### 3.4 Physicochemical Stability of Nanoparticles

### 3.4.1 Storage Stability of Nanoparticles

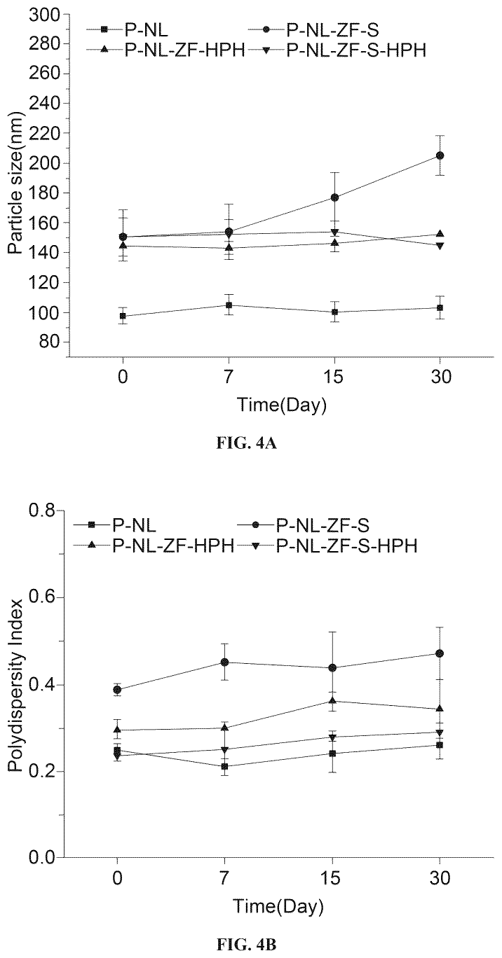

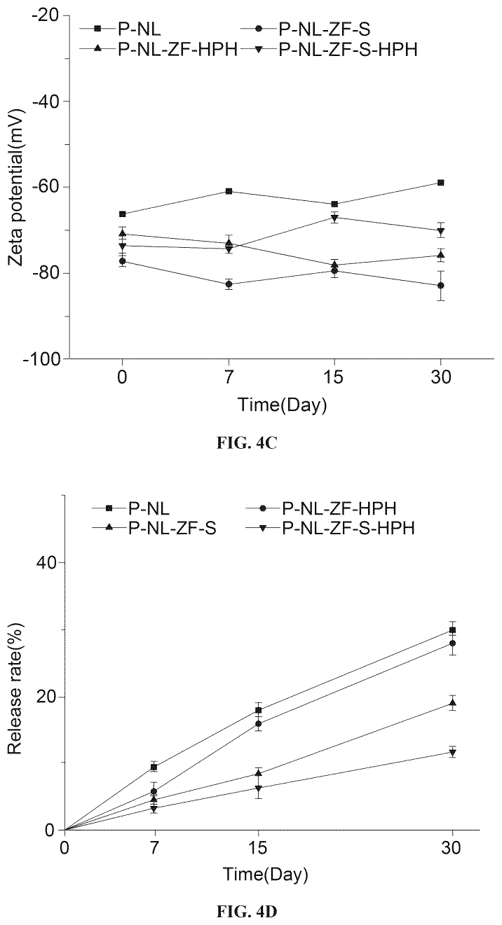

FIG. 4A illustrates the changes in particle size of different nanoparticles after being stored at 4° C. for 30 days; FIG. 4B illustrates the changes in polydispersity index (PDI) of different nanoparticles under the same storage conditions; FIG. 4C illustrates the changes in zeta potential of different nanoparticles after 30 days of storage at 4° C.; and FIG. 4D illustrates the changes in PHY release rates of different nanoparticles after the same storage duration.

FIGS. 4A-4D demonstrates the variations in particle size, PDI, zeta potential, and PHY release rates of the nanoparticles during storage. As shown in FIG. 4A, all composite nanoparticles, except for P-NL-ZF-S, maintained relatively consistent particle sizes throughout the storage period. The particle size of P-NL-ZF-S began to increase significantly after 7 days of storage, reaching 205±12.71 nm after 30 days, which represents a 26.34% increase compared to the initial preparation. Furthermore, as illustrated in FIG. 4C, the zeta potential values fluctuated within a small range as storage time extended. This fluctuation was attributed to changes in repulsive forces between particles during prolonged storage. Although the PDI of most nanoparticles exhibited minimal changes, only the P-NL-ZF-S-HPH maintained a PDI value below 0.300 after conjugation with ZF, indicating that P-NL-ZF-S-HPH exhibited homogeneity and stability over the 30-day storage period.

The influence of storage time on PHY release rates was also investigated. As shown in FIG. 4D, during the initial 15 days, the addition of ZF significantly enhanced the retention of PHY within the nanoliposomes. However, by the end of the 30-day storage period, the release rates of P-NL and P-NL-ZF-S(29.77% and 27.22%, respectively) were higher than those of P-NL-ZF-HPH (18.82%) and P-NL-ZF-S-HPH (11.67%), indicating that high-pressure treatment is more favorable for the stability of the nanoparticles. The coating of fucoidan and chitosan or pectin and chitosan on the nanoliposomes provided sufficient electrostatic repulsion, thereby enhancing the stability of the nanoliposomes. In conclusion, P-NL-ZF-S-HPH demonstrated superior physicochemical properties compared to the other treatments over an extended storage period.

### 3.4.2 Effects of pH and Salt Ions on Nanoparticle Stability

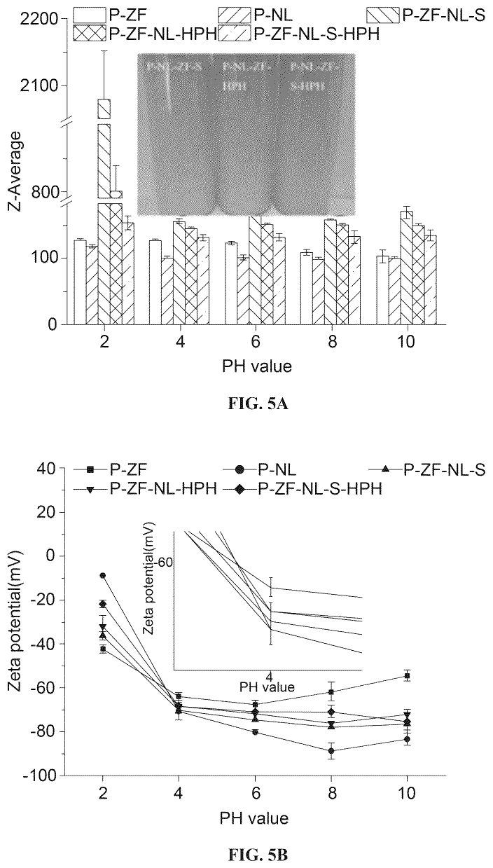

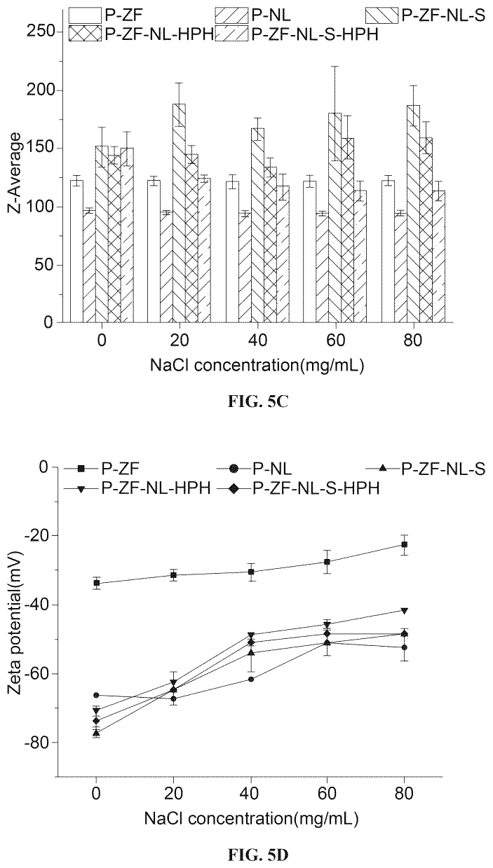

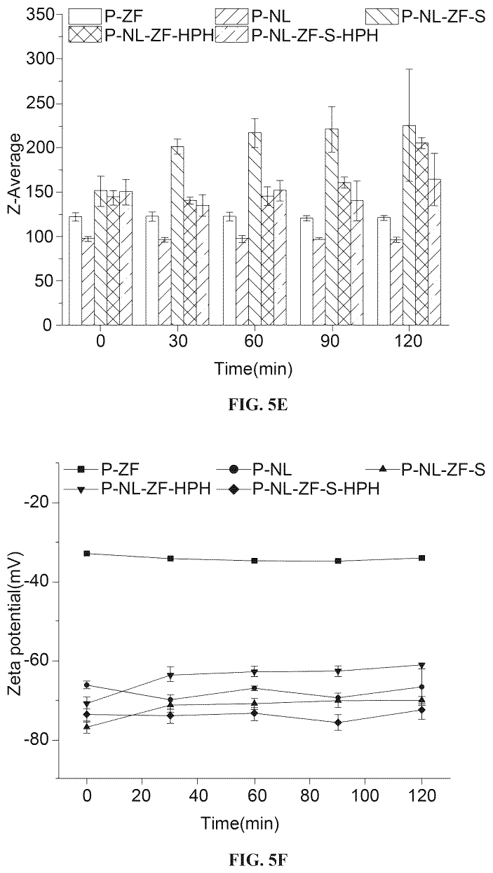

FIG. 5A illustrates the impact of different pH values (2.0, 4.0, 6.0, 8.0, and 10.0) on particle size; FIG. 5B illustrates the effects of different pH values on zeta potential; FIG. 5C illustrates the influence of NaCl concentration (0-80 mM) on particle size; FIG. 5D illustrates the effects of NaCl concentration on zeta potential; FIG. 5E illustrates the impact of thermal conditions on particle size; and FIG. 5F illustrates the effects of thermal conditions on zeta potential.

Due to the significant fluctuations in pH levels within the human gastrointestinal tract, the investigation of the pH stability of nanoparticles is considered crucial for the preparation. FIGS. 5A and 5B illustrate the changes in nanoparticle size and zeta potential at different pH levels. It was noteworthy that at a pH of 2.0, precipitation was observed in the dispersions of P-NL-ZF-S and P-NL-ZF-HPH, resulting in an increase in particle sizes to 2078.67 nm and 800.26 nm, respectively. This aggregation phenomenon was likely attributed to a reduction in surface charge and a decrease in the electrostatic repulsive forces between nanoparticles, indirectly indicating their sensitivity to acidic environments. At a pH of 3.0, aggregation of zein-fucoidan composite nanoparticles was observed, and the absolute value of the zeta potential decreased at a pH of 2.0. The alteration in charge density may lead to conformational changes, adversely affecting the stability of the nanoparticles. Therefore, P-NL-ZF-HPH and P-NL-ZF-S are deemed unsuitable for studies involving significant pH fluctuations during simulated digestion experiments. In contrast, P-NL-ZF-S-HPH exhibited remarkable anti-aggregation stability across the pH range of 2.0 to 10.0, with particle sizes ranging from 128.65 nm to 151.13 nm, underscoring the indispensable roles of magnetic stirring and high-pressure homogenization.

FIGS. 5C and 5D depict the effects of salt ions on nanoparticle stability. As the ionic strength increased, a significant expansion in the size of P-NL-ZF-S was observed. The size of P-NL-ZF-HPH decreased with increasing salt concentration at lower salt concentrations (0-40 mM) and increased at higher concentrations (40-80 mM), but aggregation did not occur. The overall trend of increasing particle size indicates that Cl⁻ and Na⁺ partially neutralized the surface charges of P-NL-ZF-S and P-NL-ZF-HPH particles, resulting in a reduction of electrostatic repulsive forces due to the shielding effect. However, particles prepared with P-NL-ZF-S-HPH were observed to have a direct decrease in size in salt solutions, suggesting that as the NaCl concentration increased, the ion saturation on the particle surfaces became higher, ultimately disrupting the shielding effect and restoring the original state. This weakening of the electrostatic shielding effect diminished the surface charge of the nanoparticles, leading to a decrease in absolute charge value and an increase in zeta potential, as shown in FIG. 5C. Therefore, both P-NL-ZF-S and P-NL-ZF-HPH demonstrated sensitivity to salt ions, rendering them unsuitable for future studies.

### 3.4.3 Thermal Stability of Nanoparticles

The results of placing nanoparticles at 80° C. for 120 minutes are presented in FIGS. 5E and 5F. It is noteworthy that significant differences in size and charge distribution were observed between P-NL-ZF-S and P-NL-ZF-HPH before and after the thermal treatment at 80° C. These differences were attributed to a series of factors, including the reconfiguration of protein molecules, the collapse of the colloidal structure, and the potential disorder of the acyl side chains in the lipid bilayer. The P-NL-ZF-S-HPH nanoparticles exhibited relative stability in particle size and potential, with minimal changes observed in both dimensions. This observation indicated that the nanoparticles prepared by P-NL-ZF-S-HPH processes manifested excellent thermal stability through complex magnetic stirring and high-pressure homogenization.

### 3.4.4 UV Stability of Nanoparticles

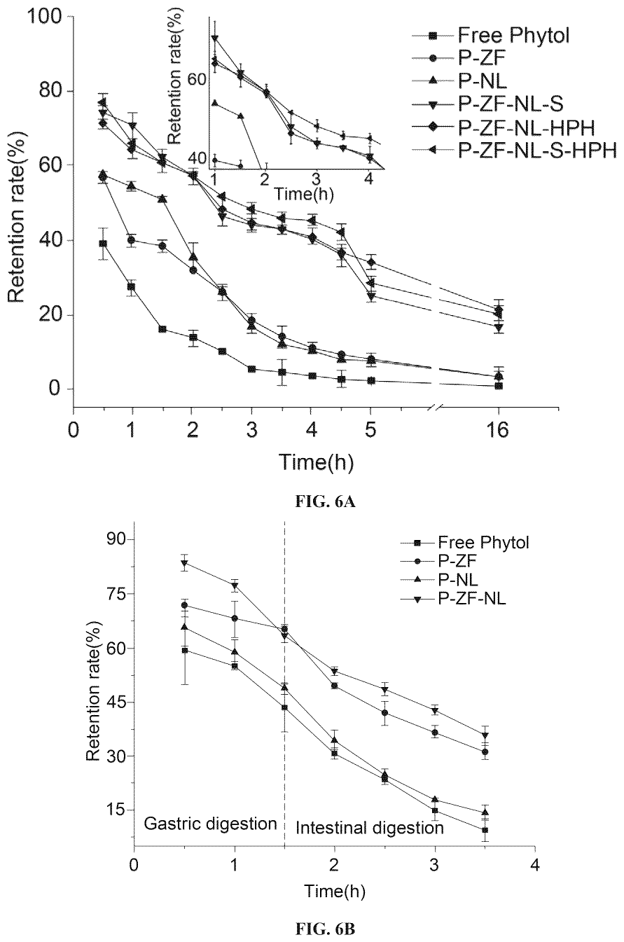

As previously mentioned, phytol is highly susceptible to degradation under ultraviolet (UV) irradiation. Therefore, a study was conducted to evaluate the ability of various delivery systems to protect PHY from photodegradation. FIG. 6A illustrates the retention rates of PHY in the nanocomplexes compared to free PHY under UV conditions, showing a gradual decline in retention rates. After 16 hours of UV exposure, the retention rates of phytol in the exposed samples and different delivery systems (P-ZF, P-NL, P-NL-ZF-S, P-NL-ZF-HPH, and P-NL-ZF-S-HPH) were 1.21%, 3.78%, 3.32%, 17.49%, 21.03%, and 20.95%, respectively. The results clearly demonstrated that the biopolymer complexes formed by ZF and P-NL provided protective effects against the photodegradation of PHY, with P-NL-ZF standing out in particular. The presence of zein endowed the nanoparticles with double bonds and aromatic amino acid residues, allowing for partial UV absorption, while the fucoidan coating served as a robust physical barrier to mitigate UV interference, thus enhancing the overall protective effect. Consequently, based on the above findings, P-NL-ZF-S-HPH was selected as the subject for subsequent studies on in vitro digestion and antioxidant activity.

### 3.5 In Vitro Digestion and Phytol Retention

Effectively controlling the release of bioactive components in the gastrointestinal digestion system is a critical evaluation criterion for food carriers. To this end, the in vitro release of phytol was investigated in simulated gastric fluid (SGF) for 90 minutes and simulated intestinal fluid (SIF) for 120 minutes. As shown in FIG. 6B, free PHY exhibited high sensitivity to the environment, as evidenced by a significant decline in retention rates: after 1.5 hours of digestion in SGF, only 44.37% of PHY remained, with nearly all remaining PHY released during the subsequent 2-hour incubation in SIF. Therefore, the use of delivery systems was employed to delay PHY degradation and reduce its release rate under gastrointestinal conditions. The loading of PHY with ZF or NL demonstrated higher retention rates, with 65.23% and 49.55% remaining in SGF, and 31.17% and 36.75% in SIF, respectively. These improvements were attributed to hydrogen bonding interactions between PHY and ZF or NL, along with the well-organized assembly of phospholipids. Furthermore, the outer FU layer provided protection against enzymatic hydrolysis of zein by gastric pepsin. Under the low pH conditions of gastric fluid, the negatively charged NL underwent protonation, leading to conformational changes in the phospholipid bilayer, resulting in sustained release. FIG. 6B illustrates the necessity and effectiveness of the combined protection offered by zein and fucoidan. The PHY encapsulated within the NL-ZF core-shell structure was effectively shielded from degradation caused by gastric acid and pepsin, achieving a retention rate of 64.81% in SGF. This compound could subsequently enter the intestine for utilization. As P-NL-ZF transitioned to the intestinal environment, changes in pH gradually induced the breakdown of the “shell” structure, leading to controlled release, as indicated by the gradual decline shown in FIG. 6B. These findings suggested that P-NL-ZF could significantly reduce the chemical degradation of PHY under simulated gastric conditions and promote its absorption in the human intestine. Notably, Mohammad Rezaul Islam Shishir et al. reported that P and CH conjugated nanoliposomes exhibited a retention rate for new naringin under simulated gastrointestinal conditions that was approximately three times that of free naringin, while the P-NL-ZF particles in this study demonstrated a retention rate for PHY under simulated gastrointestinal conditions that was 2.5 times that of P-NL, indicating a significantly superior effect compared to nanoliposomes.

### 3.6 ABTS Free Radical and Hydroxyl Radical Scavenging Activity

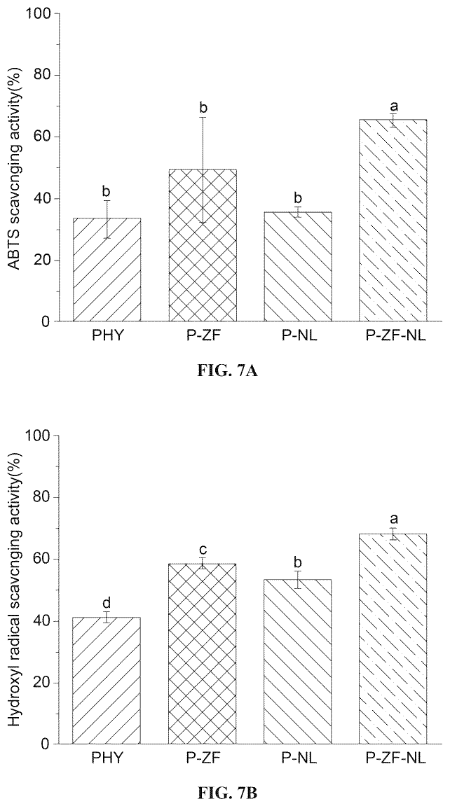

As shown in FIG. 7A, no significant differences were observed in the ABTS•+ scavenging activity between P-ZF and P-NL compared to free phytol (p>0.05). However, the scavenging capabilities of P-ZF and P-NL against •OH radicals were measured at 58% and 53%, respectively, representing increases of 45.0% and 32.5% compared to free PHY (40%). Notably, the antioxidant activity was significantly enhanced after conjugating ZF to P-NL, resulting in a 1.8-time increase compared to free PHY (p<0.05). This improvement may be attributed to the presence of aromatic amino acids in zein and the hydrogen-donating capacity of its sulfate groups. Furthermore, as discussed in previous sections, the larger surface area of the nanoparticles and the increased retention of phytol under various conditions provided more opportunities for interactions with hydroxyl radicals and ABTS radicals, thereby enhancing the scavenging efficacy.

### 3.7 In Vitro Cytotoxicity

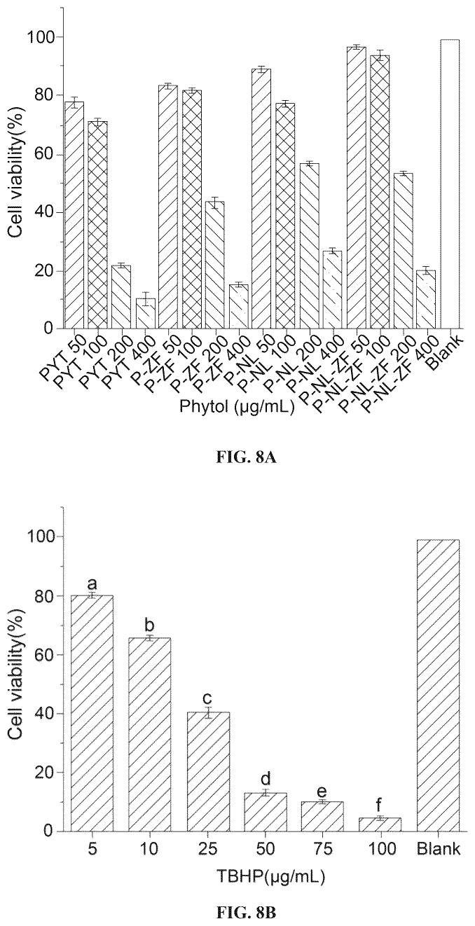

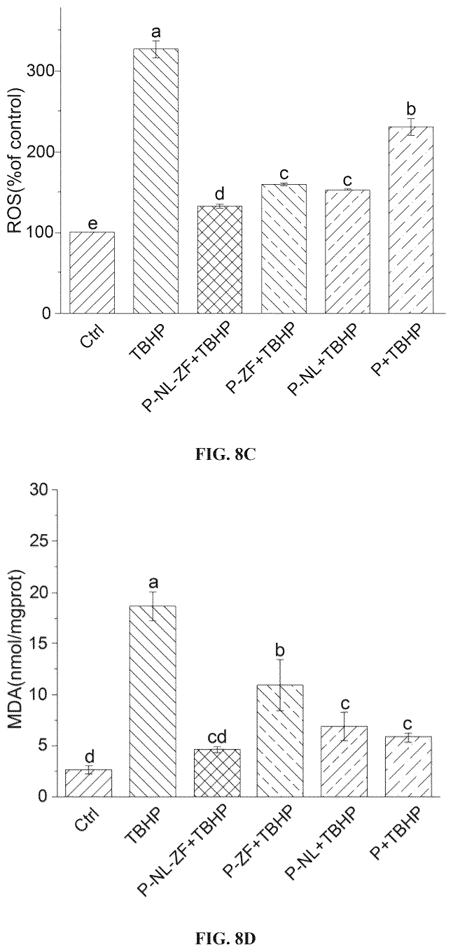

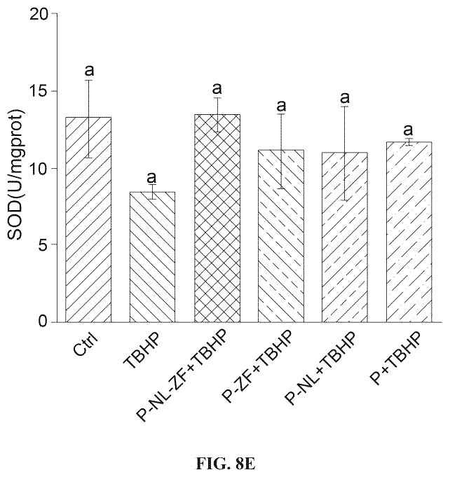

The cytotoxicity of phytol embedded in nanoparticles prepared by three different methods, as well as free phytol, was investigated against HepG2 cells using the CCK-8 assay at different concentrations (PHY concentrations: 5.00, 10.00, 25.00, 50.00, 75.00, and 100.00 g/mL). As illustrated in FIG. 8A, negligible cytotoxicity was observed within the concentration range of 50-100 μg/mL, with over 80% of cells remaining viable after 24 hours of culture. This indicated that the aforementioned concentration range can be considered non-toxic for subsequent experiments. However, within the concentration range of 200-600 g/mL, the cell viability decreased to 56-15% after 24 hours of exposure to the nanoparticle dispersion and PHY, indicating significant cytotoxicity to HepG2 cells at these concentrations. The findings suggested that the encapsulation of PHY in nanomaterials mitigated its cytotoxic effects. Therefore, 100 μg/mL phytol was selected for subsequent experiments.

Since TBHP is a known inducer of oxidative stress and cellular damage, the protective effects of PHY and the aforementioned three methods of nanoparticle preparation against TBHP-induced oxidative damage in HepG2 cells were investigated. As shown in FIG. 8B, the cell viability of HepG2 cells decreased with increasing TBHP concentrations within the range of 5-100 μg/mL, indicating that the toxicity was dose-dependent. Notably, when the TBHP concentration was 10 μg/mL, the cell viability dropped to 65.67%, demonstrating that TBHP induced significant oxidative damage. Thus, 10 μg/mL was chosen as the experimental concentration for establishing the oxidative damage model.

3.8. Influence of Nanoparticle Pre-Treatment on ROS, SOD, and MDA Levels in HepG2 Cells with TBHP-Induced Oxidative Damage.

Cellular antioxidant capacity was categorized into direct and indirect antioxidant mechanisms. Direct antioxidants provide hydrogen atoms or electrons to scavenge intracellular ROS, while indirect antioxidants mediate the expression of antioxidant enzymes and genes to combat oxidative damage.

Excessive ROS can oxidize proteins and lipids, disrupt the integrity of nuclear DNA and mitochondria, ultimately leading to cell death. DCFH-DA can penetrate cells and produce dichlorofluorescein (DCF), which binds to intracellular ROS, and generating DCFH that emits a fluorescent signal. In FIG. 8C, minimal ROS was produced in the control cells, indicating normal ROS levels. Treatment of HepG2 cells with TBHP resulted in a significant increase in ROS generation by 328.42%, indicating that TBHP induced an elevation in ROS levels. Compared to the positive control (328.16%), ROS levels were reduced by 97.44%, 168.85%, 176.14%, and 201.65% for PHY (230.72%), P-ZF (159.31%), P-NL (152.02%), and P-NL-ZF (126.51%), respectively. Although all nanoparticles significantly reduced ROS levels in HepG2 cells, the effect of P-NL-ZF was the most pronounced. Similar results were observed under fluorescence microscopy, where TBHP-stimulated cells emitted strong green fluorescence due to ROS, while PHY and the nanoparticles decreased ROS levels. These findings were consistent with results from fluorescence enzyme markers, demonstrating the antioxidant capacity of PHY Furthermore, the incorporation of nanoparticles significantly enhanced the antioxidant effect of PHY, partially attributed to the synergistic action of the carrier.

ROS generated by cells can react with polyunsaturated fatty acids in biological membranes, leading to lipid peroxidation and the formation of MDA. Both ROS and MDA are commonly used to evaluate cellular oxidative levels. The MDA levels under different treatments were depicted in FIG. 8D. HepG2 cells treated with TBHP exhibited a significant increase in MDA, indicating lipid peroxidation and oxidative damage, which may affect other macromolecules such as DNA, RNA, and proteins. All treatment methods were shown to reduce the extent of lipid peroxidation by approximately half, with the P-NL-ZF treatment demonstrating the most effective reduction.

SOD, an important endogenous antioxidant enzyme, serves as a natural superoxide scavenger in the body. It effectively removes superoxide free radicals generated during metabolism or oxidative stress. SOD activity was observed in FIG. 8E. In the damaged group treated with TBHP, SOD activity decreased to 8.43 U/mg protein. However, following treatment with PHY, P-ZF, P-NL, and P-NL-ZF, SOD activity was significantly elevated to 11.61, 11.43, 10.86, and 13.41 U/mg protein, respectively. It was noted that the components of the nanoparticles, including zein and fucoidan, also possess antioxidant capabilities, in addition to the blank nanoliposomes. The conjugation of ZF to the nanoparticles was demonstrated to produce enhanced or synergistic antioxidant effects. These results suggested that P-NL-ZF inhibits TBHP-induced oxidative stress and lipid peroxidation in HepG2 cells by reducing reactive oxygen species, enhancing antioxidant enzyme activity, and providing cellular protective functions. The improved efficacy was attributed to the antioxidant properties of the encapsulating materials (zein and fucoidan) and the increased concentration of soluble phytol.

The data presented in FIGS. 8A, 8B, 8C, 8D, and 8E are expressed as means±standard deviation, with different letters indicating significant differences (P<0.05). PHY refers to phytol; P-ZF refers to fucoidan composite nanoparticles loaded with phytol; P-NL refers to nanoliposomes loaded with phytol; and P-NL-ZF represents phytol-loaded nanoliposomes to which ZF is conjugated.

### 4. Conclusion

In this study, the physicochemical properties of phytol nanoliposomes (P-NL) and phytol nanoliposomes modified with zein and fucoidan (P-NL-ZF) were thoroughly investigated, along with their effects on the bioactivity of phytol. The increase in particle size and zeta potential, as well as the morphological changes observed in P-NL-ZF, confirmed the successful conjugation of zein and fucoidan onto the nanoliposomes. Effective interactions were formed between ZF and the lipid bilayer of the nanoliposomes. Fourier-transform infrared spectroscopy analysis revealed the presence of hydrogen bonding interactions and electrostatic interactions, further supporting these observations. Additionally, the physicochemical stability of the composite nanoparticle system was evaluated using dynamic light scattering (DLS), transmission electron microscopy (TEM), and phytol retention rate assessments. The results indicated that compared to the individual preparation methods, P-NL-ZF exhibited higher stability under varying pH, ionic strength, and temperature conditions after being subjected to both magnetic stirring and high-pressure homogenization. Similar improvements were also observed in studies on UV and storage stability, with higher phytol retention rates noted after prolonged storage (P-NL: 70.23%; P-NL-ZF-S: 72.78%; P-NL-ZF-HPH: 81.18%; P-NL-ZF-S-HPH: 88.27%) and under UV conditions (P-NL: 3.32%; P-NL-ZF-S: 17.49%; P-NL-ZF-HPH: 21.03%; P-NL-ZF-S-HPH: 20.95%). In simulated gastrointestinal experiments, the ZF biopolymer-conjugated nanoliposomes displayed an additional “protective layer” that effectively inhibited the leakage and degradation of the compound in the gastrointestinal tract, resulting in a phytol retention rate of 36.18% at the end of digestion, compared to nanoliposomes (14.19%) or zein-fucoidan nanoparticles (31.72%). Furthermore, the ZF-modified P-NL exhibited the highest antioxidant activity (ABTS radical scavenging activity, hydroxyl radical scavenging activity, superoxide dismutase activity, ROS generation, and MDA content). Cytotoxicity results indicated that P-NL-ZF dose-dependently inhibited the proliferation of HepG2 cells. These findings highlight the ZF-decorated nanoliposome system as a novel and effective delivery method, demonstrating significant advantages in the protective delivery of phytol, and suggesting great potential for the protection, delivery, and functional enhancement of other hydrophobic functional compounds.

What is claimed is:

1. A method for preparing a zein-fucoidan conjugated nanoliposome for phytol encapsulation, comprising:
(A) preparation of zein-fucoidan nanoparticles by:
i. dissolving zein in an ethanol solution with a 75% volume to volume ratio to prepare a zein stock solution with a 1% (w/v) weight to volume ratio; preparing a fucoidan solution by mixing 40 mL of deionized water with 200 mg of fucoidan, adjusting pH to 3.5; mixing 10 mL of the zein stock solution with the fucoidan solution and stirring at 1000 g for 60 minutes to obtain a mixture;
ii. removing ethanol from the mixture using a rotary evaporator, and diluting a resulting solution to 50 mL with water at pH 3.5; and
iii. centrifuging a diluted solution at 1500 g for 10 minutes to remove insoluble materials, and collecting a resulting colloidal particle dispersion, namely zein-fucoidan nanoparticles;
(B) preparation of a nanoliposome by:
i. dissolving lecithin, cholesterol, and phytol in 10 mL of ethanol, stirring to ensure complete dissolution;
ii. removing the ethanol at 45° C. and 70 rpm with a rotary evaporator to form a thin film on a container surface;
iii. placing the thin film in an oven at 30° C. to dry overnight to completely remove the ethanol;
iv. hydrating the thin film with 20 mL of ultrapure water, stirring continuously at 700 rpm and 50° C. for 1 hour; after hydration, performing ultrasonication in an ultrasonic cell disruptor in an ice bath for 15 minutes to obtain nanoliposomes that are uniform; and
v. storing the nanoliposomes at 4° C. for later use; and
(C) preparation of biopolymer-conjugated nanoliposomes by:
mixing the zein-fucoidan nanoparticles and liposomes in a molar ratio of zein to cholesterol of 1:20; using three methods for mixing, including magnetic stirring at 700 rpm for 3 hours, high-pressure homogenization at 80 bar for 3 cycles, and a combination thereof, and storing a resulting conjugated nanoliposomes at 4° C. for future use.

2. The method of claim 1, wherein the lecithin is sourced from egg yolk, with a molecular weight of 758.06 kDa and a purity greater than 80%, and the phytol has a purity of 95%.

3. The method of claim 1, wherein formulations obtained are designated as follows: P-ZF, P-NL, P-NL-ZF-S, P-NL-ZF-HPH, and P-NL-ZF-S-HPH, and wherein P represents phytol, Z represents zein, F represents fucoidan, NL represents nanoliposomes, S represents magnetic stirring, and HPH represents high-pressure homogenization.

4. A method for characterizing the zein-fucoidan conjugated nanoliposome for phytol encapsulation of claim 1, further comprising:
(D) measuring particle size and zeta potential by:
analyzing an mean particle size, polydispersity index, and (potential of composite particles by dynamic light scattering and microelectrophoresis techniques using a zetasizer; and
diluting a particle dispersion 10-fold with distilled water to mitigate a multiple scattering effect;
(E) analyzing encapsulation efficiency (EE %) by:
centrifuging a suspension of nanoparticles at 24,200 g for 22 minutes at 4° C.; separating a supernatant from solid components, and washing solid components twice with a phosphate-buffered saline (PBS) at pH 7.4; extracting phytol from both the nanoparticles and the supernatant using n-hexane, then sonicating a mixture at 30° C. for 20 minutes, and conducting quantitative analysis using gas chromatography-mass spectrometer (GC-MS) to calculate the encapsulation efficiency;
(F) assaying GC-MS by:
analyzing phytol content using a 7890A-5975C GC-MS instrument, where an Agilent J&W DB-5 ms chromatographic column has a size of 30 m×0.25 mm×0.25 μm, high-purity helium gas is used as a carrier gas at a flow rate of 1.0 mL/min, an injector temperature is set to 250° C., and a sample volume is 4.0 μL, and a splitless injection mode is used; a temperature program is started at 100° C. for 2 minutes, then increased to 250° C. at a rate of 5° C./min, and held at 250° C. for 32 minutes; an ion source temperature of the mass spectrometer is maintained at 230° C. and a transfer line temperature is set to 280° C.; a mass range of 35-600 amu, an electron energy of 70 eV and a solvent delay time of 4 minutes are set; and an obtained chromatographic ion fragment spectrum with standard ion fragments is cross-referenced for qualitative analysis of a target compound; wherein
quantification of phytol comprises weighing 0.0100 g of phytol, dissolving the phytol in n-hexane, and making up to volume in a 10 mL brown volumetric flask, diluting a solution with n-hexane to prepare standard solutions at concentrations of 1.00 mg/mL, 0.40 mg/mL, 0.20 mg/mL, 0.10 mg/mL, and 0.05 mg/mL, filtering the standard solutions through a 0.45 μm nylon membrane and analyzing by using GC-MS; calculating a linear regression equation for phytol standard samples: y=2E+09X+9E+06 (R2=0.9983) to determine an encapsulation efficiency of the phytol;
(G) observing transmission electron microscopy (TEM) by:
observing a morphology of vesicles in optimized liposomes from various batches using a high-resolution transmission electron microscope, diluting each sample with distilled water after preparation and carefully placing a single droplet onto a carbon-coated copper grid before allowing the single droplet to dry; and inverting the copper grid onto a 2% phosphotungstic acid solution and negative staining to enhance visibility of lipid components; and
(H) determining Fourier transform infrared spectroscopy (FTIR) by:
analyzing lyophilized composite nanoparticles with a FITR, with a wavelength range of 4000 to 400 cm⁻¹ for a total of 32 scans, and a resolution of 4 cm⁻¹.

5. The method of claim 4, wherein the lecithin is sourced from egg yolk, with a molecular weight of 758.06 kDa and a purity greater than 80%, and the phytol has a purity of 95%.

6. The method of claim 4, wherein formulations obtained are designated as follows: P-ZF, P-NL, P-NL-ZF-S, P-NL-ZF-HPH, and P-NL-ZF-S-HPH, and wherein P represents phytol, Z represents zein, F represents fucoidan, NL represents nanoliposomes, S represents magnetic stirring, and HPH represents high-pressure homogenization.

7. The method of claim 4, wherein evaluation of stability comprises:
(I) assessing storage stability by:
storing freshly prepared nanoparticles at 4° C. for 7, 15, and 30 days, evaluating a mean particle size and (potential of the nanoparticles afterward;
(J) assessing pH and ionic stability by:
adjusting a freshly prepared nanoparticle dispersion to pH values of 2.0, 4.0, 6.0, 8.0, and 10.0 using HCl or NaOH solutions for pH stability testing; mixing 2 mL of the freshly prepared nanoparticle dispersion with 2 mL of different concentrations of NaCl solution to prepare a series of nanoparticle dispersions with varying NaCl concentrations for ionic stability testing;
and storing all samples at 4° C. for 24 hours before measuring the size, polydispersity index (PDI), and ζ potential of the nanoparticles;
(K) evaluating thermal stability by:
heating the freshly prepared nanoparticle dispersion at 80° C. for 120 minutes, performing measurement every 30 minutes; cooling the nanoparticles to 25° C. after the heating and then measuring particle size, PDI, and surface charge; and
(L) assessing photostability by:
exposing a sample to ultraviolet (UV) light for various irradiation durations: 30 minutes, 60 minutes, 90 minutes, 120 minutes, 150 minutes, 180 minutes, 210 minutes, 240 minutes, 270 minutes, 300 minutes, and 12 hours; and determining retention of phytol content using GC-MS.

8. The method of claim 4, wherein a method of in vitro gastrointestinal digestion comprises:
conducting in vitro digestion using a simulated gastric fluid (SGF) and a simulated intestinal fluid (SIF) on both unencapsulated phytol and phytol-loaded nanoparticles, including adjusting a pH of 10 mL of the sample to 2.5 using 5M HCl before SGF treatment; mixing the sample with a SGF containing 3.2 mg/mL pepsin and 2.0 mg/mL NaCl; transferring a resulting mixture quickly to a constant temperature shaker, gently stirring at 37° C. at 120 rpm for 90 minutes; and extracting the sample every 30 minutes during a SGF digestion; and
after SGF digestion, digesting the sample by SIF, comprising adjusting the pH of the sample to 6.5 using 1M NaHCO₃ to inactivate pepsin; adding 10 mL of an SGF-digested sample to 10 mL of the SIF composed of 10 mg/mL bile salts, 3.20 mg/mL trypsin, 6.80 mg/mL K₂HPO₄, and 8.80 mg/mL NaCl; re-adjusting the pH to 7.4 using 1M NaOH before starting SIF digestion; placing the mixture in a 37° C. constant temperature shaker, gently stirring at 120 rpm for 120 minutes; and calculating the retention rate of phytol obtained from the SGF and SIF digestions.

9. The method of claim 4, wherein a method for evaluating antioxidant activity comprises:
(I) assessing scavenging activity of hydroxyl radicals by:
mixing 1.00 mL of the nanoliposomes with 1.00 mL of 9.00 mmol/L salicylic acid ethanol solution, 1.00 mL of 9.00 mmol/L FeCl2, and 1.00 mL of 8.80 mmol/L H2O2; incubating a resulting mixture in the dark at 37° C. for 30 minutes; measuring an absorbance of a reaction mixture at 536 nm using a multifunctional microplate reader and calculating a hydroxyl radical scavenging rate;
(J) assessing ABTS⁺ scavenging activity by:
storing a mixed solution of 7.00 mM 2,2′-azinobis(3-ethylbenzothiazoline-6-sulfonic acid) and 2.45 mM potassium persulfate in the dark at room temperature for 24 hours; diluting a working solution of 2,2′-azinobis(3-ethylbenzothiazoline-6-sulfonic acid) 50 times to achieve an absorbance of 0.70±0.02 at 734 nm; mixing 1.00 mL of the diluted solution with 19 mL of the working solution of 2,2′-azinobis(3-ethylbenzothiazoline-6-sulfonic acid) and performing incubation at room temperature for 1 hour; and measuring an absorbance at 734 nm and calculating the ABTS radical scavenging activity of the sample;
(K) culturing cells by:
culturing HepG2 cells in DMEM and maintaining the HepG2 cells in a cell incubator at 37° C. with a 5% CO2 atmosphere;
(L) assessing cell viability by:
determining the cell viability using a CCK-8 assay;
(M) determining tert-butyl hydrogen peroxide (TBHP)-induced oxidative damage concentration by:
seeding 1×10⁵ cells per well of digested HepG2 cells into a 96-well cell culture plate;
diluting TBHP in a serum-free Dulbecco's modified Eagle's medium (DMEM) to concentrations of 5.00, 10.00, 25.00, 50.00, 75.00, and 100.00 mg/mL; incubating the cells for 24 hours, then introducing into the plate and incubating the cells for another 24 hours, followed by determining cell viability using the CCK-8 assay;
(N) determining phytol concentration by:
seeding 1×10⁵ cells per well of the digested HepG2 cells into a 96-well cell culture plate and incubating in a cell incubator; after 24 hours of incubation, discarding a culture medium and adding different concentrations of phytol working solution to the 96-well cell culture plate; diluting a phytol stock solution in serum-free DMEM to 100.00, 200.00, 400.00, and 600.00 mg/mL, and adding the phytol working solution to the 96-well cell culture plate; continuing to incubate the cells for 24 hours, followed by determining HepG2 cell viability using the CCK-8 assay;
(O) establishing a cellular antioxidant model by:
culturing the digested HepG2 cells for 24 hours and then adding 100.00 mg/mL concentrations of PHY, P-ZF, P-NL, and P-NL-ZF to the plate for another 24 hours of incubation; removing previous culture medium and adding 5.00 mg/mL TBHP working solution to a same plate; measuring a cell survival rate, an intracellular reactive oxygen species (ROS) level, malondialdehyde (MDA) content, and superoxide dismutase (SOD) activity;
(P) measuring ROS, SOD activity, and MDA content in the cells by:
placing the digested HepG2 cells in a culture dish and performing a treatment as in step (O); adding 10.00 μM 2′,7′-dichlorodihydrofluorescein diacetate (DCFH-DA) and incubating for 0.5 hours, followed by washing with PBS; allowing intracellular esterase to cleave the DCFH-DA, which is then oxidized to highly fluorescent dichlorofluorescein in the presence of ROS; measuring a fluorescence intensity of the 96-well plate using a multifunctional microplate reader at an excitation wavelength of 485 nm and an emission wavelength of 528 nm, expressing the intracellular ROS level as a percentage of the negative control group;
assessing the SOD activity and the MDA content by culturing the digested HepG2 cells for 24 hours; after the treatment as in step (O), adding 300 μL of lysis buffer and incubating on ice for 30 minutes; centrifuging at 11,000×g for 5 minutes at 4° C. and collecting a resulting supernatant; and quantifying the SOD activity and the MDA content according to instructions of SOD and MDA assay kits; and
(Q) observing ROS production using a microscope by:
seeding the digested cells at a density of 3×10⁵ cells per well into a 6-well plate and incubating for 24 hours; after incubation, aspirating a culture medium and continuing with the grouping and treatment as in step (O); after 24 hours, replacing an original solution with 25 μM DCFH-DA solution and incubating for 60 minutes; washing the cells twice with PBS to remove unabsorbed probes; observing a fluorescence generated within the cells by placing the 6-well plate under a fluorescence microscope equipped with an FITC fluorescence channel.
